(19) United States
(12) Patent Application Publication
(10) Pub. No.: US 2024/0408121 A1
(43) Pub. Date: Dec. 12, 2024

(54) NUTRITIONAL COMPOSITION FOR IMPROVING SLEEP

(71) Applicant: SOCIETE DES PRODUITS NESTLE S.A., Vevey (CH)

(72) Inventors: NORA SCHNEIDER, Les Monts-de-Corsier (CH); FABIO MAINARDI, Lausanne (CH)

(21) Appl. No.: 18/700,600

(22) PCT Filed: Oct. 13, 2022
(86) PCT No.: PCT/EP2022/078540

§ 371 Date: Apr. 11, 2024

(30) Foreign Application Priority Data
Oct. 13, 2021 (EP) 21202427.7

(51) Int. Cl. A61K 31/685; A23L 33/00; A23L 33/12; A23L 33/15; A23L 33/16; A61K 31/202; A61K 31/519; A61K 31/688; A61K 31/714; A61K 33/26; A61P 25/20

(52) U.S. Cl. CPC A61K 31/685; A23L 33/12; A23L 33/15; A23L 33/16; A23L 33/30; A61K 31/202; A61K 31/519; A61K 31/688; A61K 31/714; A61K 33/26; A61P 25/20

(57) ABSTRACT

The present invention relates to a combination of docosahexaenoic acid, arachidonic acid, iron, vitamin B12, folic acid and phospholipids (in particular sphingomyelin, PC, PE, PI and PS) and to a nutritional composition comprising such a combination for use in improving sleep, for example in improving sleep quantity, and/or sleep quality and/or sleep efficiency and/or in promoting sleep maturation, in an infant, young child and/or child.

### FIELD OF THE INVENTION

The present invention relates to a combination of docosahexaenoic acid, arachidonic acid, iron, vitamin B12, folic acid and specific phospholipids (in particular sphingomyelin (SM), phosphatidylcholine (PC), phosphatidylethanolamine (PE), phosphatidylinositole (PI) and phosphatidylserine (PS) and to a nutritional composition comprising such a combination for use in improving sleep, for example in improving sleep quantity, and/or sleep quality and/or sleep efficiency and/or in promoting sleep maturation, in an infant, young child and/or child. The present invention also relates to a synthetic nutritional composition comprising docosahexaenoic acid, arachidonic acid, iron, vitamin B12, folic acid, sphingomyelin (SM), phosphatidylcholine (PC), phosphatidylethanolamine (PE), phosphatidylinositole (PI) and phosphatidylserine (PS) in specific amounts.

### BACKGROUND OF THE INVENTION

Breast feeding is considered as the ideal source of nutrition and is the preferred choice for feeding infants up to at least 6 months of age. Consequently, human milk (HM) has long been considered as the model for the design of infant formulas (IF). Even if many improvements in the nutrient composition of IF have been made during the last decades, there are still important differences in composition as well as in functional benefits conveyed by HM. Identifying the potential relationship between human breast milk components and early brain development and associated functions has gained substantial interests in recent years.

Nutritional deficiencies, including iron, vitamin B12, and folate, have been associated with hypomyelination, altered myelin composition, or decreased myelin synthesis. Observational studies suggest early life differences between formula-fed and breast-fed infants in fatty acid related white matter composition as well as in myelination and cognitive abilities, possibly linked to long-chain poly-unsaturated fatty acids (LC-PUFA), sphingomyelin (SM), iron and folic acid levels in infant nutrition. (Schneider N, Hauser J, Oliveira M, et al. Sphingomyelin in Brain and Cognitive Development: Preliminary Data. eNeuro. 2019; 6 (4): ENEURO. 0421-18.2019. Published 2019 Aug. 6. doi: 10.1523/ENEURO. 0421-18.2019). The role of polar lipids in neurodevelopment, possibly via myelination has been reviewed in (Zheng L, Fleith M, Giuffrida F, O'Neill BV, Schneider N. Dietary Polar Lipids and Cognitive Development: A Narrative Review. Adv Nutr. 2019 Nov. 1; 10 (6):1163-1176. doi: 10.1093/advances/nmz051. PMID: 31147721; PMCID: PMC6855982.). In our own past work (Hauser J, Sultan S, Rytz A, Steiner P, Schneider N. A blend containing docosahexaenoic acid, arachidonic acid, vitamin B12, vitamin B9, iron and sphingomyelin promotes myelination in an in vitro model. Nutr Neurosci. 2020 Dec; 23 (12): 931-945. doi: 10.1080/1028415X.2019.1580918. Epub 2019 Feb. 26. PMID: 30806182), we found that in vitro treatment with a blend of DHA, arachidonic acid (ARA), vitamin B12, folic acid, iron and sphingomyelin in a primary cell culture model increased the number of oligodendrocyte precursor cells, their differentiation and maturation.

Nonetheless, the prior art doesn't contain any information on the effect on sleep of a blend of nutrients which contains DHA, ARA, Vit B12, folic acid, iron and phospholipids.

Reduced sleep has been associated with multiple negative effects, such as decreased cognitive development, mood regulation, and overall health (L. J. Meltzer, J. A. Mindell Sleep and sleep disorders in children and adolescents Psychiatr Clin North Am, 29 (2006), pp. 1059-1076). Specifically, short sleep duration and poor sleep quality has been associated with obesity (E. M. Taveras, S. L. Rifas-Shiman, E. Oken, et al. Short sleep duration in infancy and risk of childhood overweight Arch Pediatr Adolesc Med, 162 (2008), pp. 305-311) and behavioral problems (B. Zuckerman, J. Stevenson, V.S. Bailey Sleep problems in early childhood: continuities, predictive factors, and behavioral correlates Pediatrics, 80 (1987), pp. 664-671). The most common sleep disturbances in infants and children are those related to wakefulness (i.e. either difficulties in settling at bedtime or failure to sleep through the night without interruptions). It has been estimated that these disturbances affect 15 to 35% of infants aged less than 24 months (France et al, “Infant Sleep Disturbance: Description of a problem behaviour process”, Sleep Medicine Reviews, Vol 3, No 4, pp 265-280, 1999).

To the best of our knowledge, the present study is the first study reporting human results and investigating impact of a blend of certain myelinating nutrients present in human breast milk on sleep.

There is a need to deliver such benefit on sleep in the subject in a manner that does not induce side effects and/or in a manner that is easy to deliver, and well accepted by the parents or health care practitioners. There is also a need to deliver such benefits in a manner that does keep the cost of such delivery reasonable and affordable by most.

### SUMMARY OF THE INVENTION

The present inventors have found a solution to the above-mentioned problem as set out in the appended claims.

In one aspect of the present invention, a combination of DHA, ARA, Iron, folic acid, Vitamin B12 and phospholipids is provided for use in improving sleep in an infant, young child or child.

In another aspect, a method for improving sleep in an infant, young child or child is provided comprising administering to such infant, young child or child an effective amount of a combination of DHA, ARA, Iron, folic acid, Vitamin B12 and phospholipids.

In an additional aspect, the use of a combination of DHA, ARA, Iron, folic acid, Vitamin B12 and phospholipids is provided in the manufacture of a nutritional composition for improving sleep in an infant, young child or child.

In another aspect of the present invention, a non-therapeutic use of a combination of DHA, ARA, Iron, folic acid, Vitamin B12 and phospholipids is provided for improving sleep in an infant, young child or child.

In a further additional aspect, a synthetic nutritional composition is provided which comprises sphingomyelin in an amount ranging from 300 mg/Kg to 820 mg/kg, phosphatidylcholine in an amount 500 mg to 1.3 g/Kg, phosphatidylinositol in an amount 400 mg/kg to 800 mg/kg, phosphatidylserine in an amount ranging from 200 mg/kg to 1000 mg/kg, phosphatidylethanolamine in an amount ranging from 250 mg/kg to 800 mg/kg, iron in an amount ranging from ranging from 3.5 mg/100 g to 7 mg/100 g, Vitamin B12 in an amount ranging from ranging from 0.1 mcg/100 g to 10 mcg/100 g, folic acid in an amount ranging from ranging from 110 mcg/100 g to 400 mcg/100 g and DHA and ARA in an amount 30 mg/100 g to 300 mg/100 g.

In a further aspect of the present invention a combination of DHA, ARA, Iron, folic acid, Vitamin B12 and phospholipids is provided wherein the phospholipids are phosphatidylcholine, phosphatidylinositol, phosphatidylserine, phosphatidylethanolamine and sphingomyelin and wherein such phospholipids are comprised at respective amounts ranging from 25 to 35% w/w phosphatidylcholine based on total phospholipids, amounts ranging from 5 to 15% w/w phosphatidylinositol on total phospholipids, amount ranging from 5 to 10% w/w phosphatidylserine on total phospholipids, amount ranging from 21 to 30% w/w phosphatidylethanolamine on total phospholipids and amount ranging from 20 to 30% w/w sphingomyelin on total phospholipids.

### BRIEF DESCRIPTION OF THE DRAWINGS

FIG. 1 is a Boxplots representation for night awakenings in investigational, control and breast-fed groups as described in Example 1.

FIG. 2 shows a decision tree developed as described in Example using the sleep clusters as variables to predict, and as predictors, the composition of docosahexaenoic acid, arachidonic acid, iron, vitamin B12, folic acid and sphingomyelin, plus the time-point information (1.5, 3, 6 months).

FIG. 3 is a Boxplots representation for day sleep in investigational, control and breast-fed groups as described in Example 1.

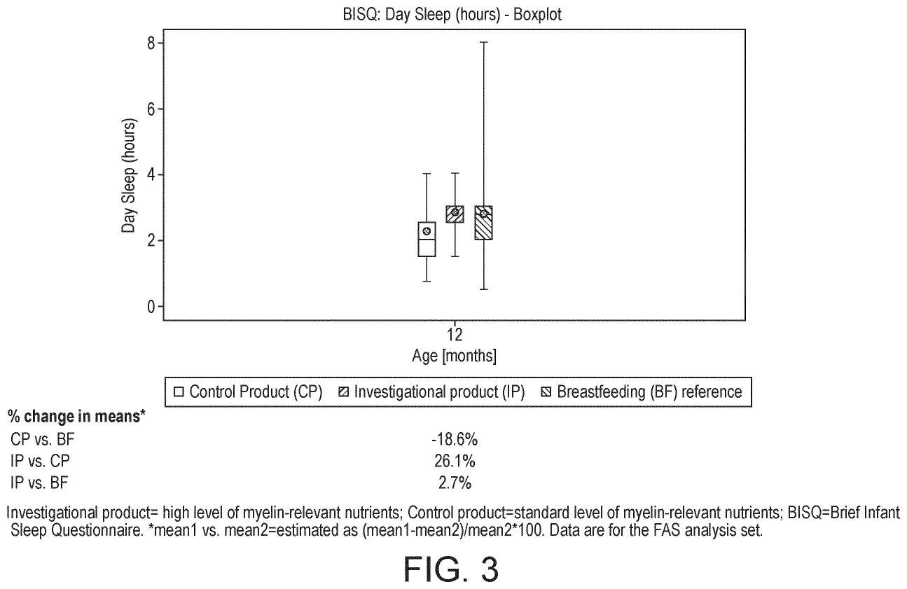

### DETAILED DESCRIPTION OF THE INVENTION

The present inventors have surprisingly found a solution to the above-mentioned problems as set out in the appended claims.

### Definitions

The term “Subject” refers to an infant, young child, child, small for gestational age (SGA), or a preterm-born child.

The term “infant” means a child under the age of 12 months. The term infant includes both infants born at term or who were born preterm.

The expression “young child” means a child aged between one and three years, also called toddler.

The expression “child” means a child aged between one and six years, including toddlers and pre-school children.

A “preterm” or “premature” means an infant, young child or child who was not born at term (40±2 weeks of gestation). Generally, it refers to an infant, young child or child born prior 38 weeks of gestation.

By the expression “small for gestational age” or “SGA” it is referred to an infant or young child who is smaller in size than normal for their gestational age at birth, most commonly defined as a weight below the 10th percentile for the gestational age. In some embodiments, SGA may be associated with Intrauterine growth restriction (IUGR), which refers to a condition in which a foetus is unable to achieve its potential size.

The “BISQ” questionnaire is the brief infant sleep questionnaire, this questionnaire has been developed to screen sleep parameters and sleep problems in infants and young children. This questionnaire has also been commonly used in sleep research to quantify quantity and quality of sleep as well as sleep maturation (Sadeh A. “Sleep assessment method” Monographs Soc Res Child Dev 80(1)2015).

In the framework of the BISQ questionnaire one or more parameters are observed and recorded that are selected in the group consisting of: nocturnal sleep duration (between the hours of 7 μm and 7 am); daytime sleep duration (between the hours of 7 am and 7 pm); number of night awaking; duration of wakefulness during the night hours (10 μm to 6 am); nocturnal sleep-onset time (the clock time at which the child falls asleep for the night); settling time (latency to falling asleep for the night); method of falling asleep; location of sleep; and preferred body position.

In the context of the present invention the term “improve sleep” indicates an amelioration in the overall sleep profile of a subject, in particular an infant and/or a young child, with respect to a control group of infants and/or young children, such overall profile being characterized, comprised or limited to the subject's sleep quality and/or to the subject's sleep quantity and/or to the subject's sleep efficiency (i.e. the ratio of total sleep time to time in bed). Such sleep quality, quantity and/or efficiency may be observed at a certain specific moment (cross-sectional observation) of the subject development and/or in the framework of a longitudinal perspective of development for the subject (i.e. sleep maturation).

Within the context of the present invention, the term “sleep quality” is characterized, comprised or is limited to sleep satisfaction as rated by caretaker, sleep problems as rated by caretaker and/or number of night awakenings. For the sake of clarity, higher sleep satisfaction and reduced sleep problems and/or night awakenings contribute to an improvement in sleep quality.

Within the context of the present invention, the term “sleep efficiency” indicates the ratio of total sleep time to time in bed.

Within the context of the present invention, the term “sleep quantity” is characterized, comprised or is limited to the length of night sleep, length of day sleep, time of total sleep duration (night & time sleep), in association to the number of episodes of wake states. For the sake of clarity, longer night sleeps and reduced number of episodes of night awakenings contribute to an improvement in sleep quantity and are an important marker of sleep maturation.

In the context of the present invention, the term “sleep maturation” or “maturation of sleeping patterns” refers to the amelioration of the sleep quality and/or quantity in the framework of a longitudinal perspective of development for the subject. In some embodiment, the term also refers to the development of a more adult-like night and day sleep pattern from a more fragmented sleep composed of frequent short bout of sleeps and absence of circadian cycle immediately after birth, to a more adult-like sleep pattern, with longer uninterrupted night sleep, limited day sleep (i.e. change of day/night sleep ratio), limited night awakening and presence of a circadian cycle.

The expression “nutritional composition” means a composition which nourishes a subject. This nutritional composition is usually to be taken orally or intravenously. It may include a lipid or fat source, a carbohydrate source and/or a protein source. In a particular embodiment the nutritional composition is a ready-to-drink composition such as a ready-to-drink formula.

In a particular embodiment the nutritional composition of the present invention is a “synthetic nutritional composition”. The expression “synthetic nutritional composition” means a mixture obtained by chemical and/or biological means, which can be chemically identical to the mixture naturally occurring in mammalian milks (i.e. the synthetic nutritional composition is not breast milk).

The expression “infant formula” as used herein refers to a foodstuff intended for particular nutritional use by infants during the first months of life and satisfying by itself the nutritional requirements of this category of person (Article 2 (c) of the European Commission Directive 91/321/EEC 2006/141/EC of 22 Dec. 2006 on infant formulae and follow-on formulae). It also refers to a nutritional composition intended for infants and as defined in Codex Alimentarius (Codex STAN 72-1981) and Infant Specialities (incl. Food for Special Medical Purpose). The expression “infant formula” encompasses both “starter infant formula” and “follow-up formula” or “follow-on formula”.

A “follow-up formula” or “follow-on formula” is given from the 6th month onwards and includes growing-up milk. It constitutes the principal liquid element in the progressively diversified diet of this category of person.

The expression “baby food” means a foodstuff intended for particular nutritional use by infants or young children during the first years of life.

The expression “infant cereal composition” means a foodstuff intended for particular nutritional use by infants or young children during the first years of life.

The term “fortifier” refers to liquid or solid nutritional compositions suitable for mixing with breast milk or infant formula.

The “mother's milk” should be understood as the breast milk or the colostrum of the mother.

Within the context of the present invention, with the term “ARA” or “AA” is intended C20:4n-6 (arachidonic acid).

Within the context of the present invention, with the term “DHA” is intended C22:6n-3 (docosahexaenoic acid).

The term “phospholipid” as used herein refers to any phospholipid, and in particular a compound of formula (1)

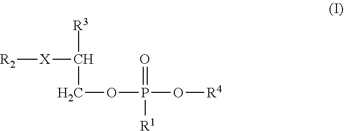

wherein,
R1 is O;
X is NH or O;
R2 is a C2-C44 saturated or unsaturated, linear or branched acyl group;
R3 is a substituent of formula (II) or formula (III):

R⁵—O—CH₂ (II)

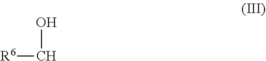

Wherein, R5 is a C2-C44 saturated or unsaturated, linear or branched acyl group and R6 is a C2-C44 saturated alkyl or alkenyl group; and
R4 is selected from; a C5 or C6 substituted or unsubstituted cyclic alkyl or alkenyl group, or,
—(CH2)ₙ-R7, wherein n is an integer ranging from 1 to 4, in particular 1 to 2 and R7 is-N(CH3)3+, NH3+, or a substituent of formula (IV) and,

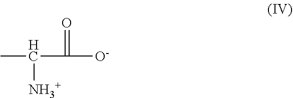

in particular R4 is a C6 cyclic alkyl or alkyl or alkenyl group substituted with one or more hydroxy groups, more particular R4 is derived from inositol (C6H12O6), and even more particularly myo-inositol i.e. R4 is:

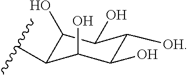 [SMILES: *C1C(O)C(O)C(O)C(O)C1O]

As used herein the term “acyclic” refers to a group that is not cyclic, i.e. does not contain a closed chain of atoms.

Phosphatidylinositol (PI) is a compound of formula (V)

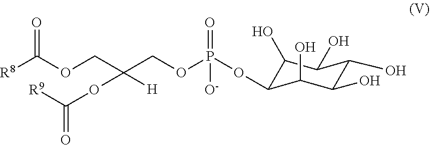

Wherein R8 is a C2 to C43 branched or unbranched acyclic alkyl, or acyclic alkenyl group and,

R9 is a C2 to C43 branched or unbranched acyclic alkyl, or acyclic alkenyl group.

More particularly R8 and R9 are, independently of each other, C13 to C43 branched or unbranched acyclic alkyl, or acyclic alkenyl groups which together with their adjacent carbonyl group are correspond to C14 to C44 saturated or unsaturated fatty acid residues, and even more particularly R8 and R9 are, independently of each other, C13 to C23 branched or unbranched acyclic alkyl, or acyclic alkenyl groups which together with their adjacent carbonyl group correspond to C14 to C24 saturated or unsaturated fatty acid residues.

More particularly, R8 and R9 are C13 to C23 branched or unbranched acyclic alkyl, or acyclic alkenyl groups which together with their adjacent carbonyl group are C14 to C24 saturated or unsaturated fatty acid residues, wherein the fatty acids from which the fatty acid residues stem are selected from the group consisting of; C14:0, C15:0, C16:0, C18:0, C20:0, C20:3, C20:4, C21:0, C22:0, C23:0, C24:0, C18:1n-9, C18:2n-6, and C24:1n-9. Even more particularly C18:0, C18:1n-9, C18:2, C20:3, and C20:4.

As the skilled person would appreciate. The term Phosphatidylserine as used herein refers to Phosphatidyl-L-serine.

Phosphatidylserine (PS) is a compound of formula (VI)

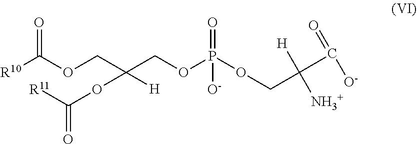

Wherein R10 is a C2 to C43 branched or unbranched acyclic alkyl, or acyclic alkenyl group and,
R11 is a C2 to C43 branched or unbranched acyclic alkyl, or acyclic alkenyl group.

More particularly, R10 and R11 are, independently of each other, C13 to C43 branched or unbranched acyclic alkyl, or acyclic alkenyl groups which together with their adjacent carbonyl group correspond to C14 to C44 saturated or unsaturated fatty acid residues, and even more particularly R10 and R11 are, independently of each other, C13 to C23 branched or unbranched acyclic alkyl, or acyclic alkenyl groups which together with their adjacent carbonyl group correspond to C14 to C24 saturated or unsaturated fatty acid residues.

More particularly, R10 and R11 are C13 to C23 branched or unbranched acyclic alkyl, or acyclic alkenyl groups which together with their adjacent carbonyl group are C14 to C24 saturated or unsaturated fatty acid residues, wherein the fatty acids from which the fatty acid residues stem are selected from the group consisting of; C14:0, C15:0, C16:0, C18:0, C20:0, C20:3, C20:4, C21:0, C22:0, C23:0, C24:0, C18:1n-9, C18:2n-6, and C24:1n-9. Even more particularly C18:0, C18:1n-9, C20:4, and C22:6.

Phosphatidylethanolamine (PE) is a compound of formula (VII)

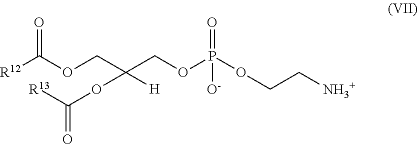

Wherein R12 is a C2 to C43 branched or unbranched acyclic alkyl, or acyclic alkenyl group and,
R13 is a C2 to C43 branched or unbranched acyclic alkyl, or acyclic alkenyl group.

More particularly, R12 and R13 are, independently of each other, C13 to C43 branched or unbranched acyclic alkyl, or acyclic alkenyl groups which together with their adjacent carbonyl group correspond to C14 to C44 saturated or unsaturated fatty acid residues, and even more particularly R12 and R13 are, independently of each other, C13 to C23 branched or unbranched acyclic alkyl, or acyclic alkenyl groups which together with their adjacent carbonyl group correspond to C14 to C24 saturated or unsaturated fatty acid residues.

The term “sphingomyelin” as used herein refers to a lipid molecule, or mixture of lipid molecules, wherein a sphingosine or a sphinganine backbone is esterified with a fatty acid residue at the amino group (—NH2) through an amide bond and wherein the hydroxyl group at position 1 of the sphingosine backbone is linked to a phosphorylcholine moiety.

In a particular sphingomyelin (SM) is a compound of formula (VIII) or a mixture of compounds of formula (VIII)

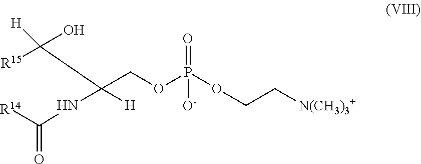

Wherein
R14 is a C2 to C43 branched or unbranched acyclic alkyl, or acyclic alkenyl group,
R15 is a C2 to C43 branched or unbranched acyclic alkyl, or acyclic alkenyl group.

More particularly, R14 is a C13 to C43 branched or unbranched acyclic alkyl, or acyclic alkenyl group which together with the adjacent carbonyl group corresponds to a C14 to C44 saturated or unsaturated fatty acid residue.

Non limiting examples of C14 to C44 saturated or unsaturated fatty acids from which the fatty acid residue may stem include; C14:0, C15:0, C16:0, C18:0, C20:0, C21:0, C22:0, C23:0, C24:1, C25:0, C28:1, C30:2, C30:1, C30:0, C32:3, C32:2, C32:1, C32:0, C33:1, C34:3, C34:2, C34:1, C34:0, C35:2, C35:0, C36:4, C36:3, C36:2, C36:1, C36:0, C37:1, C37:0, C38:4, C38:3, C38:1, C38:0, C39:1, C39:0, C40:2, C40:1, C40:0, C41:2, C41:1, C41:0, C42:47, C42:3, C42:2, C42:1, C42:0, C44:3, C44:1.

Even more particularly, R14 is a C13 to C23 branched or unbranched acyclic alkyl, or acyclic alkenyl group which together with the adjacent carbonyl group is a C14 to C24 saturated or unsaturated fatty acid residue, wherein the fatty acid from which the fatty acid residue stemmed is selected from the group consisting of; C14:0, C15:0, C16:0, C18:0, C20:0, C21:0, C22:0, C23:0, C24:0, C18:1n-9, C18:2n-6, and C24:1n-9.

Even more particularly still, sphingomyelin is a mixture of compounds of formula (VIII) wherein the mixture is such that the total number of fatty acid residues (R14 together with the adjacent carbonyl group) comprised in the mixture are predominately saturated fatty acids, and the least predominant are unsaturated fatty acids. More particularly the mixture will be such that that 80% to 96% of said fatty acid residues in the mixture are saturated fatty acids, in particular C14, C15, C16, C18, C20, C22, C23, C24 saturated fatty acids more particularly C16, C18, C20, C22 and C24.

Phosphatidylcholine (PC) is a compound of formula (IX)

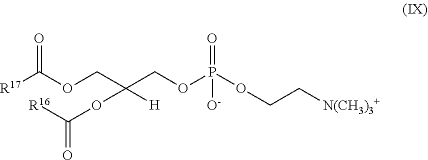

Wherein R16 is a C2 to C43 branched or unbranched acyclic alkyl, or acyclic alkenyl group and,
R17 is a C2 to C43 branched or unbranched acyclic alkyl, or acyclic alkenyl group.

More particularly, R16 and R17 are, independently of each other, C13 to C43 branched or unbranched acyclic alky, or acyclic alkenyl groups which together with their adjacent carbonyl group correspond to C14 to C44 saturated or unsaturated fatty acid residues, and even more particularly R16 and R17 are, independently of each other, C13 to C23 branched or unbranched acyclic alkyl, or acyclic alkenyl groups which together with their adjacent carbonyl group correspond to C14 to C24 saturated or unsaturated fatty acid residues.

More particularly, R16 and R17 are C13 to C23 branched or unbranched acyclic alkyl, or acyclic alkenyl groups which together with their adjacent carbonyl group are C14 to C24 saturated or unsaturated fatty acid residues, wherein the fatty acids from which the fatty acid residues stem are selected from the group consisting of; C14:0, C15:0, C16:0, C16:1, C18:0, C20:0, C20:1, C20:3, C20:4, C21:0, C22:0, C22:6, C23:0, C24:0, C18:1n-9, C18:2n-6, and C24:1n-9. Even more particularly C14:0, C16:0, C18:0, C18:1n-9, C18:2n-6, C20:1, C20:3, C20:4, and C22:6.

All percentages are by weight unless otherwise stated.

In addition, in the context of the invention, the terms “comprising” or “comprises” do not exclude other possible elements. The composition of the present invention, including the many embodiments described herein, can comprise, consist of, or consist essentially of the essential elements and limitations of the invention described herein, as well as any additional or optional ingredients, components, or limitations described herein or otherwise depending on the needs.

Any reference to prior art documents in this specification is not to be considered an admission that such prior art is widely known or forms part of the common general knowledge in the field.

### EMBODIMENTS OF THE INVENTION

The invention will now be described in further details.

It is noted that the various aspects, features, examples and embodiments described in the present application may be compatible and/or combined together.

In an embodiment the combination according to the invention comprises a phospholipid a metabolic precursor or metabolite thereof.

Non limiting examples of phospholipids include phosphatidylinositole, phosphatidylserine, phosphatidylethanolamine, sphingomyelin and phosphatidylcholine.

In an embodiment of the present invention the phospholipid is selected from the group consisting of: phosphatidylcholine, phosphatidylinositol, phosphatidylserine, phosphatidylethanolamine, sphingomyelin and/or combinations thereof.

In an embodiment of the present invention, the phospholipid comprises phosphatidylcholine, phosphatidylinositol, phosphatidylserine, phosphatidylethanolamine and sphingomyelin.

In another embodiment of the present invention, the phospholipid consists of phosphatidylcholine, phosphatidylinositol, phosphatidylserine, phosphatidylethanolamine and sphingomyelin.

In another embodiment of the present invention, the expression “total phospholipids” refers to the total amount of phosphatidylcholine, phosphatidylinositol, phosphatidylserine, phosphatidylethanolamine and sphingomyelin in the combination and/or nutritional composition.

In an embodiment of the present invention, the phospholipid comprises phosphatidylcholine, phosphatidylinositol, phosphatidylserine, phosphatidylethanolamine and sphingomyelin wherein sphingomyelin is comprised in an amount ranging from 20 to 30% w/w sphingomyelin based on total phospholipids.

In an embodiment of the present invention, the phospholipid comprises phosphatidylcholine, phosphatidylinositol, phosphatidylserine, phosphatidylethanolamine and sphingomyelin which are comprised at respective amounts ranging from 25 to 35% w/w phosphatidylcholine based on total phospholipids, amounts ranging from 5 to 15% w/w phosphatidylinositol on total phospholipids, amount ranging from 5 to 10% w/w phosphatidylserine on total phospholipids, amount ranging from 21 to 30% w/w phosphatidylethanolamine on total phospholipids and amount ranging from 20 to 30% w/w sphingomyelin on total phospholipids.

In one embodiment of the present invention, the phospholipid consists of phosphatidylcholine, phosphatidylinositol, phosphatidylserine, phosphatidylethanolamine and sphingomyelin which are comprised at respective amounts ranging from 25 to 35% w/w phosphatidylcholine based on total phospholipids, amounts ranging from 5 to 15% w/w phosphatidylinositol on total phospholipids, amount ranging from 5 to 10% w/w phosphatidylserine on total phospholipids, amount ranging from 21 to 30% w/w phosphatidylethanolamine on total phospholipids and amount ranging from 20 to 30% w/w sphingomyelin on total phospholipids.

In an embodiment of the present invention, the phospholipid comprises phosphatidylcholine, phosphatidylinositol, phosphatidylserine, phosphatidylethanolamine and sphingomyelin which are comprised at respective amounts of 32% w/w phosphatidylcholine on total phospholipids, 11% w/w phosphatidylinositol on total phospholipids, 8% w/w phosphatidylserine on total phospholipids, 25% w/w phosphatidylethanolamine on total phospholipids and 24% w/w sphingomyelin on total phospholipids.

In an embodiment of the present invention, the phospholipid consists of phosphatidylcholine, phosphatidylinositol, phosphatidylserine, phosphatidylethanolamine and sphingomyelin which are comprised at respective amounts of 32% w/w phosphatidylcholine on total phospholipids, 11% w/w phosphatidylinositol on total phospholipids, 8% w/w phosphatidylserine on total phospholipids, 25% w/w phosphatidylethanolamine on total phospholipids and 24% w/w sphingomyelin on total phospholipids.

In one embodiment of the present invention, the synthetic nutritional composition comprises total phospholipids in an amount of at least 150 mg/100 g of the synthetic nutritional composition. In one embodiment of the present invention, the synthetic nutritional composition comprises total phospholipids in an amount of at least 200 mg/100 g of the synthetic nutritional composition.

In one embodiment of the present invention, the synthetic nutritional composition comprises total phospholipids in an amount ranging from 200 to 400 mg/100 g of the synthetic nutritional composition.

In one embodiment, the synthetic nutritional composition comprises sphingomyelin in an amount higher than 200 mg/kg of the dry weight of the composition, more particularly ranging from 200 mg to 2.5 g/kg of the dry weight of the composition.

In an embodiment the synthetic nutritional composition comprises sphingomyelin in an amount selected from the group consisting of; higher than 200 mg/kg, higher than 300 mg/kg, ranging from 200 mg to 2.5 g/kg, ranging from 200 mg to 2 g/kg, in amount ranging from 300 mg to 1.5 g/kg or from 400 mg to 1 g/Kg, ranging from 200 to 850 mg/kg, or 300 to 820 mg/kg. All weights being per dry weight of the composition.

In one embodiment, the composition will comprise phosphatidylcholine in an amount higher than 200 mg/kg of the dry weight of the composition, more particularly ranging from 200 mg to 2.5 g/kg of the dry weight of the composition.

In an embodiment the synthetic nutritional composition comprises phosphatidylcholine in an amount selected from the group consisting of; higher than 200 mg/kg, higher than 300 mg/kg, higher than 400 mg/kg, ranging from 200 mg to 2.5 g/kg, ranging from 200 mg to 2 g/kg, in amount ranging from 300 mg to 1.5 g/kg or from 400 mg to 1 g/Kg, 500 mg to 1.3 g/Kg. All weights being per dry weight of the composition.

In one embodiment, the synthetic nutritional composition will comprise phosphatidylinositol in an amount higher than 500 mg/kg of the dry weight of the composition, more particularly ranging from 200 mg to 1.5 g/kg of the dry weight of the composition.

In an embodiment the composition comprises phosphatidylinositol in an amount selected from the group consisting of; higher than 200 mg/kg, higher than 300 mg/kg, ranging from 200 mg to 2.5 g/kg, ranging from 200 mg to 2 g/kg, in amount ranging from 250 mg to 800 mg/kg or from 400 mg to 1.5 g/Kg, or from 400 to 800 mg/kg. All weights being per dry weight of the composition.

In one embodiment, the synthetic nutritional composition will comprise phosphatidylserine in an amount higher than 50 mg/kg of the dry weight of the composition, higher than 200 mg/kg of the dry weight of the composition, more particularly ranging from 150 mg to 1.5 g/kg of the dry weight of the composition, from 200 mg to 1 g/kg of the dry weight of the composition

In an embodiment the synthetic nutritional composition comprises phosphatidylserine in an amount selected from the group consisting of; higher than 150, higher than 200 mg/kg, higher than 300 mg/kg, ranging from 200 mg to 2.5 g/kg, ranging from 200 mg to 2 g/kg, in amount ranging from 200 mg to 1000 mg/kg or from 250 mg to 1 g/Kg. All weights being per dry weight of the composition.

In one embodiment, the composition will comprise phosphatidylethanolamine in an amount higher than 150 mg/kg of the dry weight of the composition, higher than 200 mg/kg of the dry weight of the composition, more particularly ranging from 150 mg to 1.5 g/kg of the dry weight of the composition.

In an embodiment the composition comprises phosphatidylethanolamine in an amount selected from the group consisting of; higher than 170 mg/kg, higher than 180 mg/kg, higher than 200 mg/kg, ranging from 200 mg to 2.5 g/kg, ranging from 200 mg to 2 g/kg, in amount ranging from 250 mg to 800 mg/kg or from 200 mg to 1 g/Kg. All weights being per dry weight of the composition.

If a metabolic precursor and/or metabolite of one or more phospholipid is used in a composition in place of or in combination with a phospholipid, said compounds may be used in amounts such that the level of phospholipids physiologically delivered by said composition is in line with those set out hereinabove. It is well within the purview of the skilled person to determine appropriate amounts.

The term metabolic precursor and/or metabolite of one or more phospholipid as used herein does not include choline.

The phospholipid, metabolic precursors and/or metabolite thereof, comprised in the composition of the invention may be natural, synthetic or a mixture thereof. Said metabolic precursors and/or a metabolite, may be used in the composition of the invention in their pure form, or substantially pure form. Alternatively, they may be added in the form of a source comprising them.

Any source of a phospholipid metabolic precursors and/or metabolite thereof, suitable for ingestion by a subject for which the composition is intended to be consumed may be used in the invention.

In particular, the phospholipid a metabolic precursor or metabolite thereof, will come from natural sources, non-limiting examples of which include, eggs, soy, bovine brains, and/or mammalian milk or extracts thereof. Non limiting examples of soy sources include soy lecithin-food additive, non-limiting examples of mammalian milk include bovine, camel, sheep, goat milk including skilled milks. Non limiting extracts of milk include protein extracts e.g. whey protein and casein, milk fat globule membranes (MFGM) and extracts comprising them.

A particularly useful source of a phospholipids a metabolic precursor or metabolite thereof, in particular sphingomyelin, that may be used in the present invention may be a bovine milk whey protein concentrate enriched in alpha-lactalbumin, and/or non-pure alpha-lactalbumin which has been extracted from milk whey protein, in particular bovine milk whey protein.

Alpha-Lactalbumin is a high-quality, easy-to-digest e.g. by human infants, whey protein and is the primary protein found in HM. Alpha-lactalbumin and/or an alpha-lactalbumin enriched milk fraction is ideal for use in lower protein infant formulas due to its high content of essential amino acids, particularly tryptophan.

Although alpha-Lactalbumin is in itself a protein non pure sources may comprise sphingomyelin.

In an embodiment a phospholipid a metabolic precursor or metabolite thereof, in particular sphingomyelin, is used in the form of a whey protein concentrate enriched in alpha-lactalbumin or as alpha-lactalbumin.

In a more particular embodiment, a bovine whey protein concentrate enriched in alpha-lactalbumin or alpha-lactalbumin having a phospholipid content, in particular sphingomyelin content higher than 500 mg/100 g, 900 mg/100 g, 1000 mg/100 g dry weight of the composition is used.

Another particularly useful source of phospholipids a metabolic precursor, or metabolite thereof, may be milk fat globule membrane (hereinafter MFGM) or extracts comprising them, in particular MFGM, or extracts comprising them from bovine milk. It may be particularly beneficial if the MFGM or extracts comprising them comprises at least 1%, 2%, 5%, 10%, 20%, 30%, 40% phospholipids and/or at least 0.1%, 0.2%, 0.5% to 5%, 0.8% to 3%, 1% to 2%, 1.6%, 1.9%, 1.8% of phosphatidylcholine, phosphatidylinositol, phosphatidylserine, phosphatidylethanolamine, and/or sphingomyelin. The MFGM may also further comprise magnesium, phosphorus and or calcium, in particularly in concentrations ranging from 0.05% to 2%, 0.1% to 0.4%.

In an embodiment, the combination according to the present invention comprise Iron.

In particular, iron may be comprised in the nutritional composition in an amount higher than 5 mg/100 g of the dry composition.

In an embodiment, the nutritional composition according to the present invention comprise Iron in an amount selected from the group consisting of; higher than 4 mg, higher than 9 mg, ranging from 5 to 40 mg, ranging from 9 to 40 mg, ranging from 5 and 20 mg, ranging from 9 to 20 mg, ranging from 5 to 15 mg, ranging from 9 to 15 mg, ranging from 3.5 to 7 mg, wherein all weights are per 100 g of the dry composition.

Iron may be incorporated in the combination and/or nutritional compositions of the invention in the form of one physiologically acceptable salt such as, for example: ferric citrate, ferric phosphate, ferric pyrophosphate, ferrous ascorbate, ferrous carbonate, ferrous citrate, ferrous fumarate, ferrous gluconate, ferrous lactate, ferrous sulfate or mixtures thereof.

Iron may be incorporated in the combination and/or nutritional composition of the invention in the form of a physiologically acceptable iron complex (such as for example EDTA ferric sodium salt) or mixtures thereof.

Fe2+ is more bioavailable and it may therefore be more beneficial if iron is added into the composition in the form of a ferrous salt or complex e.g. a ferrous salts listed hereinabove.

In an embodiment, the nutritional composition according to the present invention comprises levels of iron such that the total daily intake derived from the nutritional composition of the invention will not exceed 40 mg.

In particular vitamin B12 may be comprised in the nutritional composition in an amount of selected from the group consisting of; higher than 0.01 mcg, in particular higher than 0.04 mcg, in particular higher than 0.05 mcg, wherein all weights are/100 g of the dry composition.

In an embodiment the nutritional composition of the invention comprises vitamin B12 in an amount selected from the group consisting of; higher than 0.01 mcg, higher than 0.5 mcg, higher than 0.7, higher than 5, ranging from 0.1 to 10 mcg, 0.4 to 5 mcg, 0.5 to 2 mcg, 1 to 1.5 mcg, 4 to 8.5 mcg, 5 to 8 mcg, wherein all weights are per 100 g of the dry composition.

In an embodiment, the nutritional composition according to the present invention comprises an amount of vitamin B12 such that the total daily intake derived from the nutritional composition of the invention will not exceed 7.6 mcg/100 g of the dry composition (77.6 mcg/Kg of the dry composition).

Vitamin B12 may be incorporated in the nutritional compositions of the invention as such or in the form of a physiologically acceptable salt thereof or mixtures thereof, or via any source comprising vitamin B12. In particular vitamin B12 may be incorporated into the composition in its pure form, as cyanocobalamin, hydroxocobalamin, and any combination thereof.

In particular folic acid may be comprised in an amount of higher than 50 mcg/100 g of the dry composition, more particularly 50 mcg to 500 mcg/100 g of the dry composition.

In an embodiment the nutritional composition of the invention comprises folic acid in an amount selected from the group consisting of; higher than 50 mcg, higher than 65 mcg, higher than 70 mcg, higher than 100 mcg, higher than 110 mcg, higher than 160 mcg, ranging from 50 to 500 mcg, ranging from 50 to 400 mcg, ranging from 70 to 170 mcg, ranging from 110 to 500 mcg, ranging from 110 to 400 mcg, ranging from 110 to 400 mcg, ranging from 110 to 350 mcg, wherein all weights are per 100 g of the dry composition.

In an embodiment, the nutritional composition according to the present invention comprises an amount of folic acid such that the total daily intake derived from the nutritional composition of the invention will not exceed 400 mcg.

Folic acid may be incorporated in the nutritional compositions of the invention as such or in the form of a physiologically acceptable salt thereof (folate) or mixtures thereof.

DHA and/or ARA may be comprised in the nutritional composition of the invention in an amount of 15 to 350 mg/100 g dry weight of the composition, more particularly 30 mg to 300 mg/100 g dry weight of the composition.

In an embodiment, the nutritional composition according to the present invention comprises DHA and/or ARA in an amount selected from the group consisting of; higher than 15 mg/100 g, higher than 30 mg/100 g, higher than 50 mg/100 g, higher than 55 mg/100 g, ranging from 30 to 300 mg/100 g, ranging from 30 to 200 mg/100 g or from 30 to 150 mg/100 g, ranging from 50 to 300 mg/100 g, ranging from 50 to 200 mg/100 g, ranging from 50 to 150 mg/100 g, ranging from 150 to 350, ranging from 60 to 350 mg/100 g, ranging from 60 to 120 mg/100 g, ranging from 100 to 110 mg/100 g. All concentrations are by dry weight of the composition.

Fatty acid derivatives comprising DHA and/or ARA are present in natural sources such as for example egg, algae, fungus or fish oil, algae, and in plants. Oils comprising fatty acid derivatives comprising DHA and/or ARA and generally other polyunsaturated fatty acids (PUFAs), in particular EPA (eicosapentaenoic acid), may be of various origin. Preferably, fatty acid derivatives comprising DHA are provided in the form of a fish oil comprising fatty acid derivatives comprising DHA and/or ARA. Fish oils generally comprise 5 wt. % or more, preferably 10 wt. % or more of fatty acid derivatives comprising DHA and/or ARA. Oils comprising substantial amounts of fatty acid derivatives comprising DHA and/or ARA, obtained from algae or microorganisms in general are also available. For example, oils harvested from algae comprising 10 wt. % or more, for example 20 wt. % or more of fatty acid derivatives, may be used.

ARA and DHA may for example be comprised in the composition of the invention in amounts resulting in a weight ratio of DHA: ARA in the range of 4:1 to 1:4, for example 3:1 to 1:3, for example 2:1 to 1:2, for example 1.5:1 to 1:1.5, in particular 1.1:1 to 1:1.1.

Further, when high amounts of fatty acid derivatives comprising DHA and/or ARA are comprised in the composition of the invention, it may be particularly beneficial if the total amount of fatty acid derivatives comprising saturated long chain fatty acids, in particular C20/24 is increased. These saturated long chain fatty acids may be an important component of myelin enabling it to wrap around and enrobe axons. The weight ratio of DHA and/or AA to these unsaturated long fatty acids in the composition of the invention may for example be within the range 1:11:10; 1:2 to 1:9, 1:3 to 1:4.5, 1:3.5 to 1:4.5.

The nutritional composition according to the invention can be for example an infant formula, a starter infant formula, a follow-on or follow-up formula, a baby food, an infant cereal composition, a fortifier such as a human milk fortifier, or a supplement. In an embodiment, the composition of the invention is an infant formula, a fortifier or a supplement that may be intended for the first 4 or 6 months of age. In a preferred embodiment the nutritional composition of the invention is an infant formula.

In some other embodiments the nutritional composition of the present invention is a fortifier. The fortifier can be a breast milk fortifier (e.g. a human milk fortifier) or a formula fortifier such as an infant formula fortifier or a follow-on/follow-up formula fortifier.

When the nutritional composition is a supplement, it can be provided in the form of unit doses.

The nutritional composition of the present invention can be in solid (e.g. powder), liquid or gelatinous form.

In one embodiment of the present invention, the nutritional composition is a synthetic nutritional composition.

In one embodiment of the present invention a synthetic nutritional composition is provided which comprises sphingomyelin in an amount ranging from 300 to 820 mg/kg, phosphatidylcholine in an amount 500 mg/Kg to 1.3 g/Kg, phosphatidylinositol in an amount 400 mg/Kg to 800 mg/kg, phosphatidylserine in an amount ranging from 200 mg/Kg to 1000 mg/kg, phosphatidylethanolamine in an amount ranging from 250 mg/Kg to 800 mg/kg, iron in an amount ranging from ranging from 3.5 mg/100 g to 7 mg/100 g, Vitamin B12 in an amount ranging from ranging from 0.1 mcg/100 g to 10 mcg/100 g, folic acid in an amount ranging from ranging from 110 mcg/100 g to 400 mcg/100 g and DHA and ARA in an amount 30 mg/100 g to 300 mg/100 g.

In one embodiment of the present invention, the combination and/or nutritional composition improves sleep in a subject at 3 months. In one embodiment of the present invention, the combination and/or nutritional composition improves sleep in a subject at 3 months when administered during the first 3 months of life of the subject. In one embodiment of the present invention, the combination and/or nutritional composition improves sleep in a subject at 6 months. In one embodiment of the present invention, the combination and/or nutritional composition improves sleep in a subject at 3 months when administered during the first 3 months or 6 months of life of the subject.

In one embodiment of the present invention, the combination and/or nutritional composition improves sleep in a subject at 12 months. In one embodiment of the present invention, the combination and/or nutritional composition improves sleep in a subject at 3 months when administered during the first 3, 6 or 12 months of life of the subject.

In one embodiment of the present invention, the combination and/or nutritional composition improves sleep in a subject at 18 months. In one embodiment of the present invention, the combination and/or nutritional composition improves sleep in a subject at 3 months when administered during the first 3, 6, 9, 12 or 18 months of life of the subject.

In one embodiment of the present invention, the combination and/or nutritional composition improves sleep in a subject at 24 months. In one embodiment of the present invention, the combination and/or nutritional composition improves sleep in a subject at 3 months when administered during the first 3, 6, 9, 12, 18, 24 months of life of the subject.

In one embodiment of the present invention, the combination and/or nutritional composition is for use in improving sleep quantity, and/or sleep quality and/or sleep efficiency and/or in promoting sleep maturation in a subject consuming it.

In one embodiment of the present invention, the combination and/or nutritional composition is for use in reducing the number of night awakenings in a subject consuming it. In one embodiment of the present invention, the combination and/or nutritional composition is for use in reducing the number of night awakenings in a subject consuming it at 6 months. In one embodiment, the subject consumed the nutritional composition during the first 6 months of life.

In a further embodiment of the present invention, the combination and/or nutritional composition is for use in increasing the day sleep time duration in a subject consuming it. In a further embodiment of the present invention, the combination and/or nutritional composition is for use in increasing the day sleep time duration in a subject consuming it at 12 months. In one embodiment, the subject consumed the nutritional composition during the first 12 months of life.

### Experimental Section

### Example 1

A Nutritional Composition according to the present invention improves sleep

### Clinical Trial Protocol and Analysis Procedure

Design: Randomized, controlled, double blind, two-parallel-group clinical trial with a non-randomized, non-blinded arm of exclusively breast-fed infants.

Setting: Two-center, population-based; six-month staged statistical analyses of an ongoing two-year trial.

Participants: N=81 enrolled full term, neurotypical infants of both sexes were randomized into investigational group (N=42) and control group (N=39). N=108 children in the breast-fed arm served as a natural reference group.

Intervention: a nutritional composition according to the present invention (i.e an infant formula comprising a blend of docosahexaenoic acid, arachidonic acid, iron, vitamin B12, folic acid and sphingomyelin from an uniquely processed whey protein concentrate enriched in alpha-factalbumin and phospholipids (as reported in Table 1 below).

Intervention products were bovine milk-based routine infant formulas manufactured by Wyeth Nutrition, Askeaton, Ireland. The alpha-lactalbumin enriched whey protein concentrate used for the control product was almost devoid of phospholipids and sphingomyelin, while the alpha-lactalbumin enriched whey protein concentrate used in the investigational product contained higher levels of sphingomyelin and phospholipids as a result of this ingredient's unique manufacturing process. The investigational product also contained higher levels of DHA, ARA, iron (fortified through Ferrous Sulfate Heptahydrate), folic acid and vitamin B12 (fortified through Cyanocobalamine) (Table 1 and 2) than the control product.

Sleep: Sleep behavior was assessed by infant sleep questionnaire (Brief Infant Sleep Questionnaire (BISQ), Sadeh A. A brief screening questionnaire for infant sleep problems: validation and findings for an Internet sample. Pediatrics. 2004; 113 (6): e570-e577.). Sleep was assessed at 3-, 6-, 12-, 18- and 24-month visits. Additional data is expected for 12-, 18- and 24-month visits. The BISQ is a parent-report brief infant sleep screening tool capturing nocturnal sleep duration (between the hours of 7 μm and 7 am); daytime sleep duration (between the hours of 7 am and 7 μm); number of night wakings; duration of wakefulness during the night hours (10 μm to 6 am); nocturnal sleep-onset time (the clock time at which the child falls asleep for the night); settling time (latency to falling asleep for the night); method of falling asleep; location of sleep; and preferred body position. Sleep problems are rated on a 3-point-scale. The BISQ has good, demonstrated test-retest reliability and validity (Sadeh, 2004) and is applicable for infants and young children 0-3 years of age.

Outcome measures for the clinical trial include night sleep (hrs: min), day sleep (hrs: min), total sleep, no of night awakenings.

Statistics: Both intervention groups were formally compared using descriptive statistics, independent t-tests as well as ANCOVA corrected for stratification factors. The p-values were not corrected for multiplicity.

TABLE 1
Nutrient levels in study intervention groups (nutritional composition
of intervention for investigational product (IP) (according to the
invention) and control product (CP)) after reconstitution and
breastfeeding (BF) (non-randomized reference group)

| | Investigational | Control |
|---|---|---|
| | Levels | Levels |
| Energy (kcal per | 132 | 132 |
| 200 ml serving) | | |
| Sphingomyelin | 84 | 22 |
| (mg/L) | | |
| DHA (mg/L) | 128 | 71 |
| ARA (mg/L) | 132 | 71 |
| Iron (mg/L) | 6.9 | 4.0 |
| Folic Acid (mcg/L) | 175 | 85 |
| B12 (mcg/L) | 5.6 | 0.9 |
| Phospholipids | 410 | 186 |
| (mg/L) | | |

TABLE 2
Nutrient levels in study intervention groups (nutritional
composition of intervention for investigational product (IP)
(according to the invention) and control product (CP))

| | Investigational | Control |
|---|---|---|
| | Levels | Levels |
| Sphingomyelin | 65 | 17 |
| (mg/100 g) | | |
| DHA (mg/100 g) | 99 | 55 |
| ARA (mg/100 g) | 102 | 55 |
| Iron (mg/100 g) | 5.3 | 3.1 |
| Folic Acid | 136 | 65.5 |
| (mcg/100 g) | | |
| B12 (mcg/100 g) | 4.32 | 0.71 |
| Phospholipids | 318 | 144 |
| (mg/100 g) | | |

In the nutritional composition according to the invention described sphingomyelin was comprised in an amount ranging from 300 mg/Kg to 820 mg/kg, phosphatidylcholine was comprised in an amount 500 mg to 1.3 g/Kg, phosphatidylinositole was comprised in an amount 400 mg/kg to 800 mg/kg, phosphatidylserine was comprised in an amount ranging from 200 mg/kg to 1000 mg/kg and phosphatidylethanolamine was comprised in an amount ranging from 250 mg/kg to 800 mg/kg.

### Results

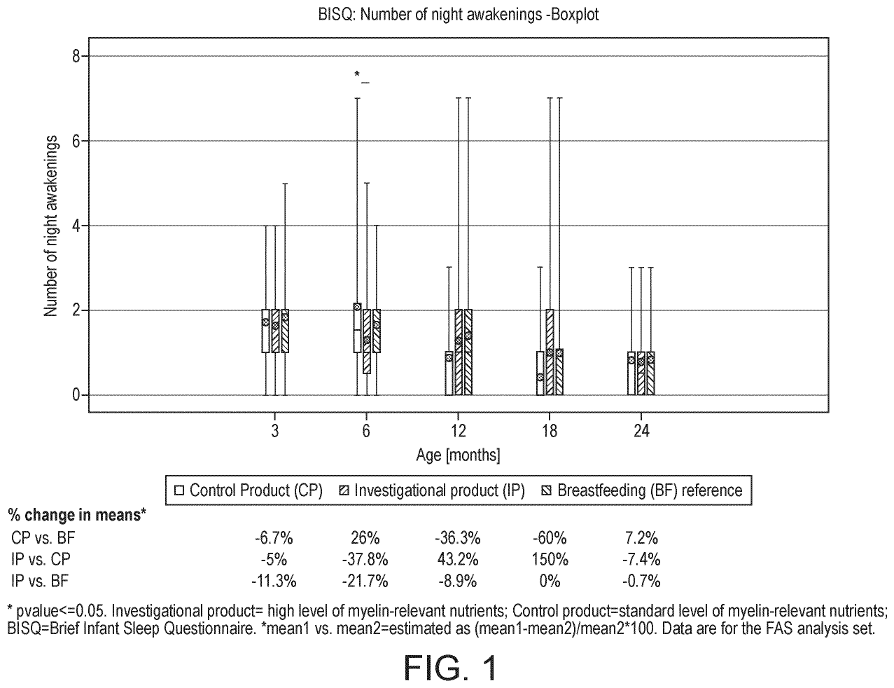

Children receiving the investigational nutrient blend slept more during the day at 12 months (2.9 vs. 2.4 hours, +0.5 hours; p=0.044) and showed significantly less night awakenings at 6 months (2.2 vs. 1.3,−40%; p=0.046), while total night sleep hours were similar between groups (Table 3 and FIG. 1 ad 3).

TABLE 3
Night awakenings

| Changes in | | | | | |
|---|---|---|---|---|---|
| Means | | | | | |
| [mean 1 vs | | | | | |
| mean 2 | | | | | |
| estimated | | | | | |
| as (mean1- | | | | | |
| mean2)/ | | | | | |
| mean | 3 | 6 | 12 | 18 | 24 |
| 2*100] | months | months | months | months | months |
| CP vs BF | 1.7 vs | 2.1 vs 1.6 | 0.9 vs 1.4 | 0.4 vs | 0.8 vs 0.76 |
| | 1.8 | | | 1 | |
| IP vs CP | 1.6 vs | 1.3 vs 2.1* | 1.3 vs 0.9 | 1 vs | 0.75 vs 0.8 |
| | 1.7 | (p = 0.046) | | 0.4 | |
| IP vs BF | 1.6 vs | 1.3 vs 1.6 | 1.3 vs 1.4 | 1 vs | 0.75 vs 0.76 |
| | 1.8 | | | 1 | |

*pvalue <= 0.05

Sleep behaviors mature rapidly in the first year of life and lower number of night awakenings as well as longer day time naps in infants have been linked to better memory performance (Lukowski et al. 2013, Infant Behav Dev. 2013; 36 (3):369-376).”

### Example 2

A Nutritional Composition according to the present invention correlates with longer night sleep

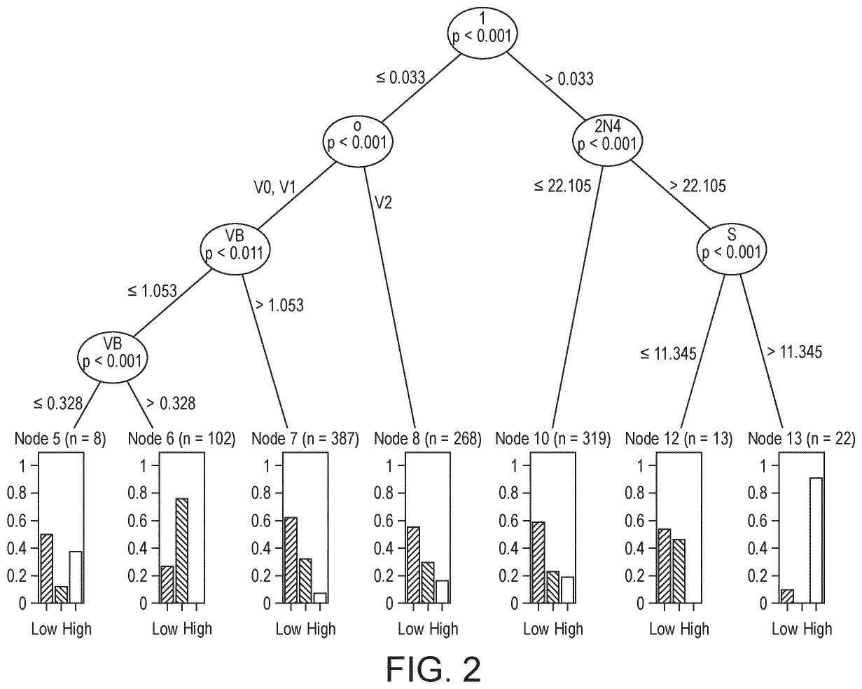

Further data analysis was performed on data from clinical trial described in Example 1. Breastmilk composition data were available at 1.5 months, 3 months and 6 months. Sleep data were available at 6, 12, 18 and 24 months. Sleep data consisted of BISQ scores for night sleep duration, day sleep duration and number of night wakenings. Functional clustering (J.-L. Wang, J.-M. Chiou and H.-G. Müller (2016). Functional data analysis. Annal Review of Statistics and Its Applications, 3, 257-95) was performed on the night sleep duration and 3 clusters were identified. Functional clusters describe longitudinal patterns of sleep evolution; for simplicity we label them has ‘low’, ‘mid’, ‘high’, based on the average duration of night sleep. A decision tree was then fit to the data (Hothorn T, Hornik K, Zeileis A (2006). “Unbiased Recursive Partitioning: A Conditional Inference Framework.” Journal of Computational and Graphical Statistics, 15 (3), 651-674), using the sleep clusters as variables to predict, and as predictors, the composition of docosahexaenoic acid, arachidonic acid, iron, vitamin B12, folic acid and sphingomyelin, plus the time-point information (1.5, 3, 6 months). The result is shown in FIG. 2.

By browsing the tree from top to bottom one reaches a node, where the estimated probability of each cluster is given. For example, the rightmost branch leads to cluster “higher sleep” with high probability, corresponding to the following rule: Iron>0.032 mg/100 mL, DHA>22.1 mg/100 mL, Sphingomyelin>11.34 mg/100 mL. Overall, the decision tree shows that a blend containing higher amounts of certain nutrients according to the present invention presents an increased probability of longer average night sleep duration as compared to blends comprising less of those nutrients or lower amounts.

It should be understood that various changes and modifications to the presently preferred embodiments described herein will be apparent to those skilled in the art. Such changes and modifications can be made without departing from the spirit and scope of the present invention and without diminishing its attendant advantages. It is therefore intended that such changes and modifications be covered by the appended claims.

1. (canceled)

2. Method according to claim 12, wherein the phospholipids are selected from the group consisting of sphingomyelin, phosphatidylcholine, phosphatidylinositol, phosphatidylserine and phosphatidylethanolamine.

3. Method according to claim 12 wherein the phospholipids comprise at respective amounts 25 to 35% w/w phosphatidylcholine based on total phospholipids, 5 to 15% w/w phosphatidylinositol on total phospholipids, 5 to 10% w/w phosphatidylserine on total phospholipids, 21 to 30% w/w phosphatidylethanolamine on total phospholipids and 20 to 30% w/w sphingomyelin on total phospholipids.

4. Method according to claim 12 for improving sleep quality, sleep quantity, sleep efficiency and/or sleep maturation.

5. Method according to claim 12 for promoting sleep maturation.

6-7. (canceled)

8. Method according to claim 12, wherein the combination is in a nutritional composition selected from the group consisting of an infant formula, a starter infant formula, a follow-on or follow-up infant formula, a baby food, an infant cereal composition, a fortifier and a supplement.

9. Method according to claim 12 wherein the combination comprises sphingomyelin in an amount ranging from 300 mg/kg to 820 mg/kg, phosphatidylcholine in an amount 500 mg/kg to 1.3 g/Kg, phosphatidylinositol in an amount 400 mg/kg to 800 mg/kg, phosphatidylserine in an amount ranging from 200 mg to 1000 mg/kg, phosphatidylethanolamine in an amount ranging from 250 mg to 800 mg/kg, iron in an amount ranging from ranging from 3.5 mg/100 g to 7 mg/100 g, Vitamin B12 in an amount ranging from ranging from 0.1 mcg/100 g to 10 mcg/100 g, folic acid in an amount ranging from ranging from 110 mcg/100 g to 400 mcg/100 g and DHA and ARA in an amount 30 mg/100 g to 300 mg/100 g.

10. A combination of DHA, ARA, Iron, folic acid, Vitamin B12 and phospholipids wherein the phospholipids are phosphatidylcholine, phosphatidylinositol, phosphatidylserine, phosphatidylethanolamine and sphingomyelin and wherein such phospholipids are comprised at respective amounts ranging from 25 to 35% w/w phosphatidylcholine based on total phospholipids, amounts ranging from 5 to 15% w/w phosphatidylinositol on total phospholipids, amount ranging from 5 to 10% w/w phosphatidylserine on total phospholipids, amount ranging from 21 to 30% w/w phosphatidylethanolamine on total phospholipids and amount ranging from 20 to 30% w/w sphingomyelin on total phospholipids.

11. A synthetic nutritional composition which comprises sphingomyelin in an amount ranging from 300 mg/Kg to 820 mg/kg, phosphatidylcholine in an amount 500 mg to 1.3 g/Kg, phosphatidylinositol in an amount 400 mg/kg to 800 mg/kg, phosphatidylserine in an amount ranging from 200 mg/kg to 1000 mg/kg, phosphatidylethanolamine in an amount ranging from 250 mg/kg to 800 mg/kg, iron in an amount ranging from ranging from 3.5 mg/100 g to 7 mg/100 g, Vitamin B12 in an amount ranging from ranging from 0.1 mcg/100 g to 10 mcg/100 g, folic acid in an amount ranging from ranging from 110 mcg/100 g to 400 mcg/100 g and DHA and ARA in an amount 30 mg/100 g to 300 mg/100 g.

12. A method for improving sleep in an infant, young child or child, the method comprising administering to such infant, young child or child an effective amount of a combination of DHA, ARA, Iron, folic acid, Vitamin B12 and phospholipids.

13-14. (canceled)
